(19) United States
(12) Patent Application Publication
(10) Pub. No.: US 2006/0204436 A1
(43) Pub. Date: Sep. 14, 2006

(54) Tritiated growth hormone secretagogue mk-0677

(76) Inventors: Cornelia Hertel, Muenchenstein (CH); Philipp Huguenin, Liestal (CH); Sannah Jensen Zoffmann, Basel (CH); Jean-Marc Plancher, Rue des Vignes (FR)

Correspondence Address: HOFFMANN-LA ROCHE INC.;PATENT LAW DEPARTMENT, 340 KINGSLAND STREET, NUTLEY, NJ, 07110, US

(21) Appl. No.: 10/561,654

(22) PCT Filed: Jun. 18, 2004
(86) PCT No.: PCT/EP04/06567

§ 371 Date: Apr. 11, 2006

(30) Foreign Application Priority Data
Jun. 25, 2003 (EP) 03013471.2

(51) Int. Cl. A61K 51/00; C07D 487/10

(52) U.S. Cl. 424/1.61; 546/16; 534/11

(57) ABSTRACT

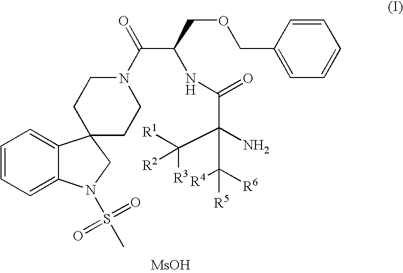

The present invention relates to a tritiated growth hormone secretagogue which can be used for the identification of compounds capable of binding to growth hormone secretagogue receptors, or of compounds having activity as growth hormone secretagogues.

Growth hormone, which is secreted from the pituitary, stimulates growth of all tissues of the body that are capable of growing. In addition, growth hormone is known to have the following basic effects on the metabolic processes of the body: (1) Increased rate of protein synthesis in all cells of the body; (2) Decreased rate of carbohydrate utilization in cells of the body; (3) Increased mobilization of free fatty acids and use of fatty acids for energy. A deficiency in growth hormone secretion can result in various medical disorders, such as dwarfism.

Various ways are known to release growth hormone. For example, chemicals such as arginine, L-3,4-dihydroxyphenylalanine (L-DOPA), glucagon, vasopressin, and insulin induced hypoglycemia, as well as activities such as sleep and exercise, indirectly cause growth hormone to be released from the pituitary by acting in some fashion on the hypothalamus perhaps either to decrease somatostatin secretion or to increase the secretion of the known secretagogue growth hormone releasing factor (GRF) or an unknown endogenous growth hormone-releasing hormone or all of these.

By the term “growth hormone secretagogue” (GHS) is meant any compound or agent that directly or indirectly stimulates or increases the release of growth hormone in an animal. Growth hormone secretagogues (especially a growth hormone secretagogue bearing a radiolabel) are useful in vitro as unique tools for understanding how growth hormone secretion is regulated at the pituitary level. This includes use in the evaluation of many factors thought or known to influence growth hormone secretion such as age, sex, nutritional factors, glucose, amino acids, fatty acids, as well as fasting and non-fasting states. In addition, growth hormone secretagogues are useful in the evaluation of how other hormones modify growth hormone releasing activity. For example, it has already been established that somatostatin inhibits growth hormone release. Other hormones that are important and in need of study as to their effect on growth hormone release include the gonadal hormones, e.g., testosterone, estradiol, and progesterone; the adrenal hormones, e.g., cortisol and other corticoids, epinephrine and norepinephrine; the pancreatic and gastrointestinal hormones, e.g., insulin, glucagon, gastrin, secretin; the vasoactive peptides, e.g., bombesin, the neurokinins; and the thyroid hormones, e.g., thyroxine and triiodothyronine. Growth hormone secretagogues may also be employed to investigate the possible negative or positive feedback effects of some of the pituitary hormones, e.g., growth hormone and endorphin peptides, on the pituitary to modify growth hormone release. Of particular scientific importance is the use of growth hormone secretagogues to elucidate the subcellular mechanisms mediating the release of growth hormone.

Methodology is known in the art to determine the activity of a compound as a growth hormone secretagogue. For example, an ex vivo assay is described by Smith, et al., Science, 260,1640-1643 (1993) (see text of FIG. 2 therein), but this assay requires the use of cell cultures and does not give an indication of competitive binding activity. Accordingly, it would be desirable to develop a radioligand which can be used to identify and characterize cellular receptors which play a role in the activity of growth hormone secretagogues. It would also be desirable to have a radioligand available for use in an assay for testing compounds for growth hormone secretagogue activity.

Such studies normally require a high specific activity radioligand. Previous attempts to develop a binding assay using [T]-labeled or [125I]-labeled peptide ligands derived from GHRP-6 met with limited success. See R. F. Walker, et al. Neuropharmacol. 989,28, 1139 and C. Y. Bowers et al., Biochem. Biophys. Res. Comm. 1991, 178,31. Generally, the binding of such peptide ligands was of low affinity and of excessively high capacity. Moreover, the binding affinities did not correlate with the growth hormone secretory activity of the peptides. The lack of correlation of binding and growth hormone secretory activity most likely was the result of the relatively low specific activity (in the case of [T] GHRP-6) and non-specific binding properties of the radioligands.

WO 9722367 provides a [35S] radiolabeled compound which stimulates the release of endogenous growth hormone and possesses high specific radiochemical activity. Said compound belongs to Spiro compounds disclosed in WO 94113696/EP0615977 and Proc. Natl. Acad. Sci. USA, 92, 7001-7005 (July 1995) as being non-peptidal growth hormone secretagogues. These compounds have the ability to stimulate the release of natural or endogenous growth hormone. Among the preferred compounds disclosed therein is N-[1(R)-[(1,2-dihydro-1-methanesulfonylspiro[3H-indole-3,4′-piperidin]-lt-yl)carbonyl]-2-(phenylmethyloxy)ethyl]-2-amino-2-methylpropanamide which has high growth hormone secretory activity. These compounds are disclosed as unlabelled compounds. The interest of the present invention consists in providing a radiolabelled compound of the type described in WO 94113696/EP0615977, for use in screening assays. Generally, in situations where specific activity >100 Ci/mmol is required, radioiodine is the label of choice for the study of receptors. See K. G. McFarthing, In Receptor-Ligand Interactions: A Practical Approach; Hulme, E. C., Ed.; Oxford University Press, Oxford, 1992; Chapter 1. However, incorporation of a halogen atom (e.g. Cl, Br) at the para position of the benzyl group or in the 5-position of the Spiro-indoline phenyl group of spiro[3H-indole-3,4′-piperdin]-1′-yl)carbonyl]-2-(phenylmethyl-oxy)ethyl]-2-amino-2-methylpropanamide lead to a >20-fold loss in intrinsic activity on growth hormone release from rat pituitary cells. This indicated that iodine substitution (such as with 125I) at these positions would not afford a high potency ligand. Moreover, iodine has a high degree of lipophilicity and could further alter the affinity of the ligand. Enhanced hydrophilicity (water solubility) is usually inversely related to the observed “stickiness” of the radioligand which often severely restricts its usefulness in various receptor preparations, see M. W. Cunningham, et al. In Radioisotopes in Biology: A Practical Approach; Slater, R. J., Ed.; Oxford University Press, Oxford, 1990; Chapter 6. Note in this regard that ligands bearing the methane sulfonamide group should exhibit reduced lipophilicity (X value of NHSO, CH,=−1.18) compared with 125I congeners (X value of 1=1.12). See C. Hansch, et al. J. Med. Chem. 1973, 16, 1207. In addition the amino functionality was found to be essential for biological activity and so conjugating it with the widely used Bolton-Hunter reagent was not a viable alternative. See K. G. McFarthing, In Receptor-Ligand Interactions: A Practical Approach; Hulme, E. C., Ed.; Oxford University Press, Oxford, 1992; Chapter 1.

In WO 9722367 a N-[1(R)-[(1,2-dihydro-1-methanesulfonylspiro[3H-indole-3,4′-piperidin]-lt-yl)carbonyl]-2-(phenylmethyloxy)ethyl]-2-amino-2-methylpropanamide radiolabelled with [35S] to a high specific activity was achieved.

The main disadvantages of using 35S labelled radioligands are, however, the short half life and the requirements for waste diposal. As already indicated above, previous attempts to develop a binding assay using Tritium-labeled peptide ligands derived from GHRP-6 met with limited success. Surprisingly, it was found by the present inventors that a tris-tritiated-2-amino-N-[(R)-2-[1,2-dihydro-1-(methylsulfonyl)spiro[3H-indole-3,4′-piperidin]-1′-yl]-2-oxo-1-[(phenylmethoxy)methyl]ethyl]-2-methyl-, monomethanesulfonate could be provided with a sufficiently high specific activity that it can be successfully used in binding assays.

### DESCRIPTION OF THE INVENTION

The present invention relates to a radio-labeled growth hormone secretagogue comprising at least one compound of the formula 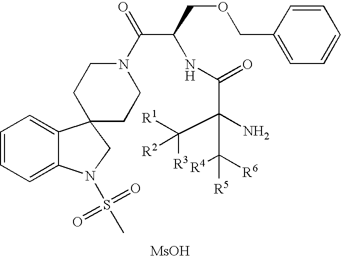
wherein R¹ to R⁶ are independently of each other H or T, and wherein at least one of R¹ to R⁶ are T.

The term “radiolabeled growth hormone secretagogue” as used herein refers to individual compounds hereinbefore described as well as to mixtures of compounds hereinbefore described. Thus, the radiolabeled growth hormone secretagogue of the present invention may also be characterized by an average number of T present per molecule of said compound.

The term “T” as used herein refers to a Tritium atom.

The term “tris-tritiated-2-amino-N-[(1R)-2-[1,2-dihydro-1-(methylsulfonyl)spiro[3H-indole-3,4′-piperidin]-1′-yl]-2-oxo-1-[(phenylmethoxy)methyl]ethyl]-2-methyl-, monomethanesulfonate”, refers to a radioligand hereinbefore described wherein an average of about three T is present in said radioligand.

The term “MK0677” refers to the unlabeled compound of the formula indicated above, wherein R¹ to R⁶ are H. The term “T-MK0677” as used herein refers to the Tritium-radiolabeled growth hormone secretagogue of the present invention.

The structure of the unlabeled compound has been disclosed in patent EP 0615977.

In a preferred embodiment, the radiolabeled growth hormone secretagogue hereinbefore described has a specific activity of between 86.4 Ci/mmole and 115.2 Ci/mmole. In a more preferred embodiment, the radiolabeled growth hormone secretagogue hereinbefore described has a specific activity of 97.5 Ci/mmole.

The present invention provides a use of a radiolabeled growth hormone secretagogue hereinbefore described for identifying a compound that can bind to a growth hormone secretagogue receptor. A use of a radiolabeled growth hormone hereinbefore described for identifying a cellular receptor as a growth hormone secretagogue receptor is also provided. In addition, the radiolabeled growth hormone secretagogue hereinbefore described can be used for identifying the activity of a compound as a growth hormone secretagogue.

Furthermore, the present invention provides a process of synthesizing a radiolabeled growth hormone secretagogue which comprises reacting a compound having a formula: 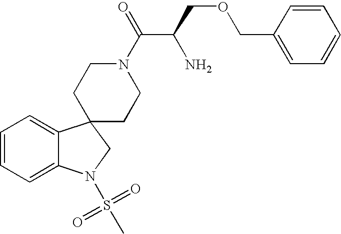
with X-α-aminoisobutyric acid-[methyl-T], where X is defined as a protecting group which is subsequently removed if present and salts are formed if desired.

The present invention also relates to a radiolabeled growth hormone secretagogue obtainable by the process hereinbefore described.

Further to this, the present invention pertains to a method of identifying a cellular receptor expressed in a host as a growth hormone secretagogue receptor comprising contacting the cellular receptor with the radiolabeled growth hormone secretagogue hereinbefore described and determining whether binding of said radiolabeled growth hormone secretagogue has occurred. The host may be a tissue, a primary cell or a cultured cell suspected of expressing a growth hormone secretagogue receptor.

The present invention provides a method for identifying a compound that can bind to a growth hormone secretagogue receptor comprising contacting said compound with a host expressing a growth hormone secretagogue receptor in the presence of the radiolabeled growth hormone secretagogue hereinbefore described and monitoring whether the compound influences the binding of the radiolabeled growth hormone secretagogue to the growth hormone secretagogue receptor. The host may be a tissue sample, primary cells or cultured cells which either naturally express a growth hormone secretagogue receptor, or which are either transiently or stably transfected with a growth hormone secretagogue receptor. Methods of transfecting cells are well known in the art (Sambrook et al., Molecular Cloning: A Laboratory Manual (1989), Cold Spring Harbor Laboratory Press, New York, USA).

Furthermore, the present invention also provides a method for identifying the activity of a compound as a growth hormone secretagogue comprising contacting the compound suspected of having activity as a growth hormone secretagogue with a host expressing a growth hormone secretagogue receptor in the presence of the radiolabeled growth hormone secretagogue hereinbefore described and monitoring whether the compound suspected of having activity as a growth hormone secretagogue influences the binding of the radiolabeled growth hormone secretagogue hereinbefore described to the growth hormone secretagogue receptor. The host may be a tissue sample, primary cells or cultured cells which either naturally express a growth hormone secretagogue receptor, or which are either transiently or stably transfected with a growth hormone secretagogue receptor. Methods of transfecting cells are well known in the art (Sambrook et al., Molecular Cloning: A Laboratory Manual (1989), Cold Spring Harbor Laboratory Press, New York, USA).

The present invention also provides a compound identified by the methods hereinbefore described or pharmaceutically acceptable salts thereof. In addition, the present invention provides a pharmaceutical composition comprising a compound hereinbefore described and a pharmaceutically acceptable carrier.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the identified agents wherein the parent agent is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, benzenesulfonic, toluenesulfonic, methanesulfonic, ethane disulfonic, oxalic, isethionic, and the like.

The pharmaceutically acceptable salts of the present invention can be synthesized from the parent agent which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

The agents identified by the method of the invention may be modified to achieve (i) modified site of action, spectrum of activity, and/or (ii) improved potency, and/or (iii) decreased toxicity (improved therapeutic index), and/or (iv) decreased side effects, and/or (v) modified onset of action, duration of effect, and/or (vi) modified kinetic parameters (resorption, distribution, metabolism and excretion), and/or (vii) modified physico-chemical parameters (solubility, hygroscopicity, color, taste, odor, stability, state), and/or (viii) improved general specificity, organ/tissue specificity, and/or (ix) optimized application form and route by (i) esterification of carboxyl groups, or (ii) esterification of hydroxyl groups with carbon acids, or (iii) esterification of hydroxyl groups to, e.g. phosphates, pyrophosphates or sulfates or hemi succinates, or (iv) formation of pharmaceutically acceptable salts, or (v) formation of pharmaceutically acceptable complexes, or (vi) synthesis of pharmacologically active polymers, or (vii) introduction of hydrophilic moieties, or (viii) introduction/exchange of substituents on aromates or side chains, change of substituent pattern, or (ix) modification by introduction of isosteric or bioisosteric moieties, or (x) synthesis of homologous compounds, or (xi) introduction of branched side chains, or (xii) conversion of alkyl substituents to cyclic analogues, or (xiii) derivatisation of hydroxyl group to ketales, acetales, or (xiv) N-acetylation to amides, phenylcarbamates, or (xv) synthesis of Mannich bases, imines, or (xvi) transformation of ketones or aldehydes to Schiffs bases, oximes, acetales, ketales, enolesters, oxazolidines, thiozolidines or combinations thereof; and (b) formulating the product of said modification with a pharmaceutically acceptable carrier or a carrier/diluent acceptable for fragrance or flavor compositions or products.

Any conventional carrier material can be utilized. The carrier material can be an organic or inorganic one suitable for eteral, percutaneous or parenteral administration. Suitable carriers include water, gelatin, gum arabic, lactose, starch, magnesium stearate, talc, vegetable oils, polyalkylene-glycols, petroleum jelly and the like. Furthermore, the pharmaceutical preparations may contain other pharmaceutically active agents. Additional additives such as flavoring agents, stabilizers, emulsifying agents, buffers and the like may be added in accordance with accepted practices of pharmaceutical compounding.

The present invention also pertains to the radiolabeled ligand, compounds, methods, process, uses and composition substantially as hereinbefore described, especially with reference to the following examples.

### SHORT DESCRIPTION OF THE FIGURES

FIG. 1 shows a Mass Spectrometry result of the analysis of the end product, tris-tritiated-2-amino-N-[(1R)-2-[1,2-dihydro-1-(methylsulfonyl)spiro[3H-indole-3,4′-piperidin]-1′-yl]-2-oxo-1-[(phenylmethoxy)methyl]ethyl]-2-methyl-, monomethanesulfonate. The term [3H] refers to Tritium (T).

FIG. 2A: A binding curve of T-MK0677 to Human Embryonic Kidney HEK 293 (EBNA) cell membranes is shown. The term [3H] refers to Tritium (T).

FIG. 2B: Competition of binding of T-MK0677 to Human Embryonic Kidney HEK 293 (EBNA) cell membranes by different growth hormone secretagogue receptor antagonists is shown. The term [3H] refers to Tritium (T).

### EXAMPLES

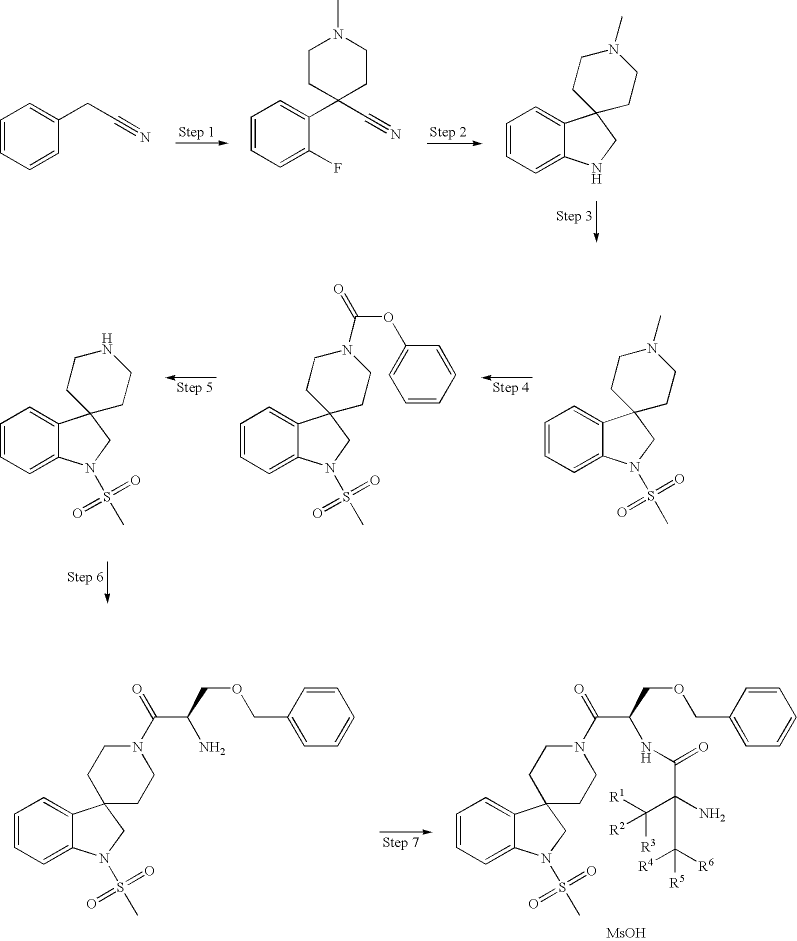

### Example 1.1

### Preparation of the 4-(2-Fluoro-phenyl)-1-methyl-piperidine-4-carbonitrile

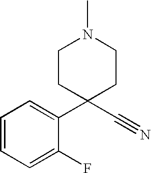

To a solution of 2-fluorophenylacetonitrile (5 g, 37 mmol, 1.16 eq.) in DMSO (75 mL) was added NaH dispersion (55% in oil, 5.92 g, 148 mmol, 4.65 eq.), with vigorous stirring. After 30 min, a solution of 2,2′-dichloro-N-methyldiethylamine.hydrochloride (6.12 g, 31.8 eq. 1 eq.) in DMSO (75 mL) was added dropwise, and the mixture was stirred at 75° C. for 4 h30. Ice water (300 g) was then added and the mixture was extracted with diethylether. The combined ether solution was shaken with hydrochloric acid (2N). Organic layer was discarded. The aqueous layer was basified with sodium hydrogenocarbonate (84 g) and extracted with diethylether. After evaporation of the solvents, the resulting oil was purified with flash chromatography. One fraction was isolated, evaporated and dried in vacuo, yielding 3.0 g (43%) of 4-(2-Fluoro-phenyl)-1-methyl-piperidine-4-carbonitrile, as a brown oil. ISP-MS: m/e=218.1 ([M]⁺).

### Example 1.2

### Preparation of the 1′-methylspiro(indoline-3,4′-piperidine)

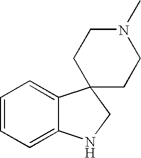

To a cooled (0° C.) suspension of lithium aluminium hydride (2.09 g, 55 mmol, 4.0 eq.) in dimethoxyethane (70 mL) was slowly added dry ethanol (7.6 mL, 165 mmol, 12 eq.). Following addition, the mixture was slowly heated to reflux. A solution of 4-(2-Fluoro-phenyl)-1-methyl-piperidine-4-carbonitrile in dimethoxyethane (30 mL) was added over a 30 min-period. Reflux was maintained during 72 h. The reaction mixture was cooled down to room temperature and decomposed with water (2.2 mL), aqueous solution of sodium hydroxide (15%, 2 mL) and finally, water (7 mL).

After 30 min, the mixture was filtered and the filter cake was washed twice with warm dichloromethane. The filter cake was dried in vacuo, yielding 2.12 g (76%) of 1′-methylspiro(indoline-3,4′-piperidine) as a yellow solid. ISP-MS: m/e=203.1 ([M+H]⁺).

### Example 1.3

### Preparation of the N-mesyl-N′-methylspiro(indoline-3,4′-piperidine)

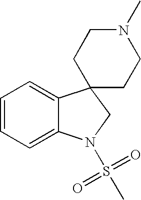

To a cooled (0° C.) solution of 1′-methylspiro(indoline-3,4′-piperidine) in dichloromethane (40 mL) was slowly added methanesulfonic acid chloride in solution in dichloromethane, (10 mL) within 30 min. After 2 h20 at 0° C., the solution was diluted with dichloromethane (150 mL), poured unto an aqueous solution of sodium hydrogenocarbonate (250 mL), washed with brine, dried over sodium sulfate, filtered and then dried in vacuo. 2.85 g of N-mesyl-N′-methylspiro(indoline-3,4′-piperidine) were obtained as a yellow semisolid. ISP-MS: m/e=281.2 ([M+H]⁺).

### Example 1.4

### Preparation the of the N-mesyl-N′-carboxyphenoxyspiro(indoline-3,4′-piperidine)

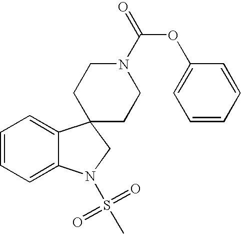

A solution of of N-mesyl-N′-methylspiro(indoline-3,4′-piperidine) and phenylchloroformate in dichloromethane was stirred for 20 h at 20° C. The reaction mixture was washed with an aqueous of sodium hydroxyde (10%) and then water. After concentration, purification was performed by flash chromatography. One fraction was isolated, evaporated and dried in vacuo, yielding 3.0 g of N-mesyl-N′-carboxyphenoxyspiro(indoline-3,4′-piperidine) as a white solid. ISP-MS: m/e=387.2 ([M+H]⁺).

### Example 1.5

### Preparation of the Spiro[3H-indole-3,4′-piperidine], 1,2-dihydro-1-(methylsulfonyl)

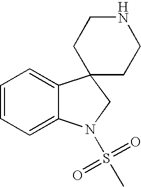

A solution of N-mesyl-N′-carboxyphenoxyspiro(indoline-3,4′-piperidine) (3.0 g, 7.76 mmol) and potassium hydroxyde (6.0 g, 90.9 mmol, 11.7 eq.) in ethylene glycol (45 mL) was stirred at 160-170° C. under nitrogen for 105 min. The reaction mixture was cooled down to room temperature, diluted with ice-water (400 mL) and extracted with dichloromethane. The organic layer was washed with water and concentrated in vacuo, yielding 1.87 g of spiro[3H-indole-3,4′-piperidine], 1,2-dihydro-1-(methylsulfonyl) as a white solid. ISP-MS: m/e=267.2 ([M+H]⁺).

### Example 1.6

### Preparation of the Spiro[3H-indole-3,4′-piperdine], 1′-[(2R)-2-amino-1-oxo-3-(phenylmethoxy)propyl]-1,2-dihydro-1-(methylsulfonyl)

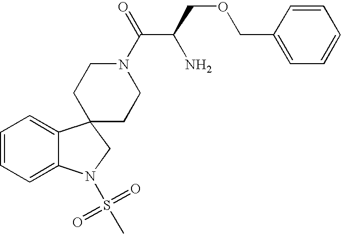

Water (20 mL), dicyclohexylcarbodiimide (1.576 g, 7.64 mmol, 1.1 eq.) and 1-hydroxybenzotriazole (1.02 g, 7.57 mmol, 1.09 eq.) and Boc-O-Benzyl-D-serine (2.26 g, 7.64 mmol, 1.10 eq.) were added to a solution of spiro[3H-indole-3,4′-piperidine], 1,2-dihydro-1-(methylsulfonyl) (1.85 g, 6.94 mmol) in isopropylacetate (50 mL). The mixture was stirred 5 h at 20° C., then filtered.

The solid was filtered and washed with isopropylacetate (30 mL). The aqueous layer was separated, and the organic phase was washed with an aqueous solution of sodium hydroxyde (1M, 30 mL), an aqueous solution of hydrochloric acid (0.5 M, 2×30 mL) and an aqueous solution of sodium hydrogenocarbonate (30 mL). The organic layer was evaporated, yielding 3.94 g of a white light solid. This solid was diluted with ethanol (16 mL). Methane sulfonic acid (1.35 mL, 20.8 mmol, 3.0 eq.) was added. The mixture was a warmed at 40° C. for 7.5 h.

Water (50 mL) was added. The mixture was cooled down to 5° C. for 30 min and filtered. The pH was adjusted to pH>12 by adding an aqueous solution of sodium hydroxyde (3M, 7.5 mL). The mixture was extracted with isopropylacetate. After concentration, the crude mixture was purified by flash chromatography. One fraction was collected, evaporated and dried in vacuo, yielding 2.32 g of spiro[3H-indole-3,4′-piperidine], 1′-[(2R)-2-amino-1-oxo-3-(phenylmethoxy)propyl]-1,2-dihydro-1-(methylsulfonyl) as a white solid. ISP-MS: m/e=444.2 ([M+H]⁺).

### Example 1.7

### Preparation of the tris-tritiated 2-amino-N-[(1R)-2-[1,2-dihydro-1-(methylsulfonyl)spiro[3H-indole-3,4′-piperidin]-1′-yl]-2-oxo-1-[(phenylmethoxy)methyl]ethyl]-2-methyl-, monomethanesulfonate

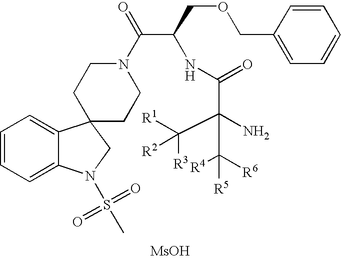 with R¹ to R⁶ being independently of each other H ot T.

An ethanolic solution of 88.51 mCi of N-Boc-α-aminoisobutyric acid-[methyl-T], (100 Ci/mmole, International Isotopes Clearing House, Inc., Leawood, Kans. USA), was filtered and transferred into a 0.3 mL reactor. The solvent was evaporated under argon. N,N′-dicyclohexylcarbodiimide (0.206 mg) and 1-hydroxybenzotriazole (0.135 mg), were added to a solution of Spiro[3H-indole-3,4′-piperidine], 1′-[(2R)-2-amino-1-oxo-3-(phenylmethoxy)propyl]-1,2-dihydro-1-(methylsulfonyl) (0.444 mg) in isopropyl acetate. Water was added (13.5 μL). The mixture was stirred vigorously at room temperature for 2 h.

The reaction mixture was diluted with ethyl acetate and washed with saturated sodium hydrogencarbonate solution. The organic solution was dried over sodium sulfate and the solvents were evaporated in vacuo. A solution of methanesulfonic acid in ethanol (0.4 mL, 5% v/v) was added. This solution was stirred overnight at 40° C.

The same work-up as described above furnished 47.9 mCi of the desired product with 77% radiochemical purity according to HPLC.

Purification by HPLC (column: Zorbax Bonus RP 5 μm) yielded 17.78 mCi of tris-tritiated-2-amino-N-[(1R)-2-[1,2-dihydro-1-(methylsulfonyl)spiro[3H-indole-3,4′-piperidin]-1′-yl]-2-oxo-1-[(phenylmethoxy)methyl]ethyl]-2-methyl-, monomethanesulfonate with 99.5% radiochemical purity according to HPLC.

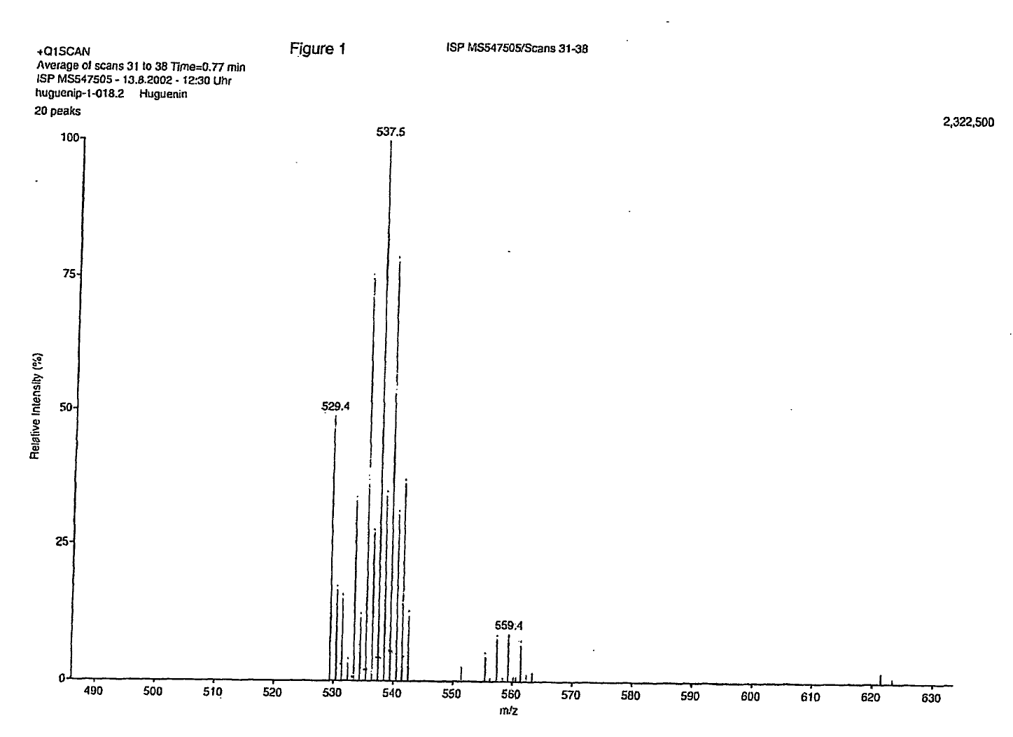

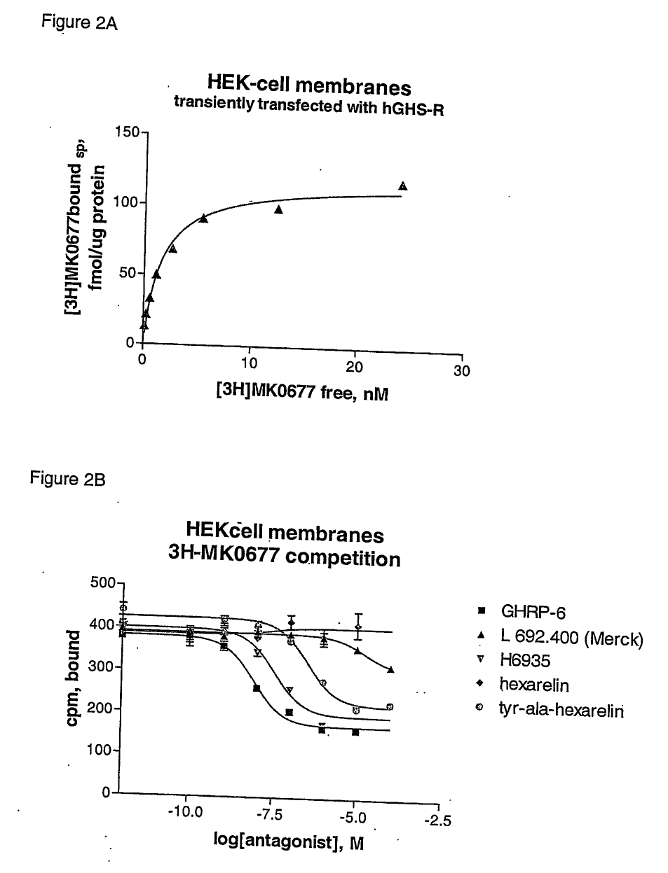

The specific activity of the solid reaction product was: 97.5 Ci/mmole (determined by mass spectrometry, see FIG. 1).

### Example 2

### Binding Assay

### Example 2.1

### Membrane Preparation

Human Embryonic Kidney HEK 293 (EBNA) cells were grown in suspension and transfected according to the method previously described (Schlaeger and Christensen, Cytotechnology, 30, 71-83, 1999). The cells were centrifugated for 10 min at 500 rpm, washed once with PBS-0.7 mM EDTA/(4° C.) and resuspended in PBS-EDTA-PI (with Protease inhibitor cocktail), at 2 ml/g of cells. Cells were broken with Ultra Turax level green 3×15″ with 30″ breaks on ice. To remove debris the suspension was centrifugated in a Sorvall SS34 rotor for 20 min at 2,000 rpm. The supernatant was collected and centrifugated for 40 min at 20,000 rpm. The pellet was resuspended in PBS-EDTA. Receptor density was verified with saturation binding assay using T-MK 0677 to be 4.9 pmol/mg protein.

### Example 2.2

### Binding Assay

The membrane was resuspended in Binding buffer (25 mM Hepes, pH 7.4, 25 mM MgCl₂, 1 mM CaCl₂, 0.1% BSA, 0.03% Bacitracine (protease inhibitor)) to 0.1 to 2 μg/well.

Binding buffer, T-MK0677 (10 mM, 97.5 Ci/mmol from Amersham, to a final concentration of 10 nM), unlabeled ligand (to a final concentration of 1 μM) and resuspended membrane (final concentration 0.5 μg/well) were added to a final volume of 200 μl/well to a 96 well microtiter plate (Corning 3600 non-binding surfaces). The mixture was incubated for 60 min at 20° C.

A filter plate (Packard GF/B unifilter plate) was treated with 50 μL/well 0.5% polyethyleneimine (PEI) in PBS for 15 min. The binding mixture was filtered through the filter plate, the filter plate was washed three times with icecold PBS. The filter plate was then dried in a preheated incubator at 50° C. for 50 min. Then 50 μL of Microscint 0 were added per well and the T decay counted in a topcount from Packard Instruments.

1. A radiolabeled growth hormone secretagogue comprising at least one compound of the formula: 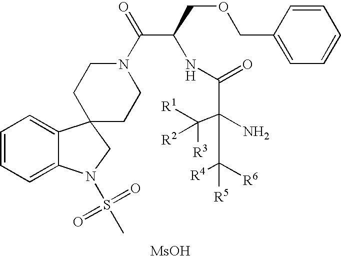
wherein R¹ to R⁶ are independently of each other H or T, and wherein at least one of R¹ to R⁶ are T.

2. The radiolabeled growth hormone secretagogue of claim 1, wherein the specific activity of the radiolabeled growth hormone secretagogue is between 86.4 Ci/mmole and 115.2 Ci/mmole.

3. The radiolabeled growth hormone secretagogue of claim 2, wherein the specific activity of the radiolabeled growth hormone secretagogue is 97.5 Ci/mmole.

4-7. (canceled)

8. A method of identifying a cellular receptor as a growth hormone secretagogue receptor comprising contacting a host suspected to express a growth hormone secretagogue receptor with the radiolabeled growth hormone secretagogue of claim 1 and determining whether binding has occurred.

9. A method for identifying a compound that can bind to a growth hormone secretagogue receptor comprising contacting said compound with a host expressing a growth hormone secretagogue receptor in the presence of the radiolabeled growth hormone secretagogue of claim 1 and monitoring whether the compound influences the binding of said radiolabeled growth hormone secretagogue to the growth hormone secretagogue receptor.

10. (canceled)

11. A compound identified by the method of claim 9, or pharmaceutically acceptable salts thereof.

12. A pharmaceutical composition comprising a compound of claim 11 and a pharmaceutically acceptable carrier.

13. (canceled)
